(19) United States
(12) Patent Application Publication
(10) Pub. No.: US 2013/0323713 A1
(43) Pub. Date: Dec. 5, 2013

(54) SUN TRACKING LIGHT DISTRIBUTOR SYSTEM HAVING A V-SHAPED LIGHT DISTRIBUTION CHANNEL

(71) Applicant: INSTITUT NATIONAL D'OPTIQUE, Quebec (CA)

(72) Inventor: Marc LEVESQUE, Saint-Augustin-de-Desmaures (CA)

(73) Assignee: INSTITUT NATIONAL D'OPTIQUE, Quebec (CA)

(21) Appl. No.: 13/780,857

(22) Filed: Feb. 28, 2013

Related U.S. Application Data
(60) Provisional application No. 61/655,517, filed on Jun. 5, 2012.

(51) Int. Cl. C12M 1/00

(52) U.S. Cl. CPC C12M 41/08
USPC 435/3; 435/286.2

(57) ABSTRACT

A sun-tracking light distributor system for use in one of an open-ended system and a closed photo-bioreactor for a photosynthetic culture having an aqueous liquid. The sun-tracking light distributor system comprises two light distribution walls, provided at an angle and oriented to converge at a bottom end, the light distribution walls being made of a transparent material allowing sunlight to pass therethrough, the two converging light distribution walls creating an elongated V-shaped channel with an interior space adapted to receive the sunlight and an exterior surface adapted to be partly immersed in the aqueous liquid in use; and a displacement system operatively connected to at least one of the two light distribution walls, the displacement system being adapted to change an orientation of at least one of the two light distribution walls to track a solar position with respect to at least one axis.

### TECHNICAL FIELD

The invention relates to light distributors for use in open and closed photosynthetic culture aqueous systems which use the sun as a light source, for example for the production of algae. The light distributors have a V-shaped light distribution surface and are adapted to be oriented with a displacement system to track the sun and provide a distribution of the light in the aqueous volume.

### BACKGROUND OF THE ART

Photosynthetic culture in aqueous liquids is often used for the production of algae. Two main types are known in the art, the open-ended systems such as ponds and basins and the closed systems such as photo-bioreactors (PBR). The aqueous liquid provided in the system typically includes water and the photosynthetic culture organisms such as algae or micro-organisms. It can include other substances.

The prior art discusses different configurations to allow light to penetrate the aqueous liquid of the aqueous systems. These configurations either do not take into account the solar position throughout the day and throughout the year or can exhibit prohibitive optical losses or alignment precision requirements.

There is a need to improve light distribution in open-ended and closed aqueous systems to benefit from exposure to as much light as possible from the sun.

### SUMMARY

According to one broad aspect of the present invention, there is provided a sun-tracking light distributor system for use in one of an open-ended system and a closed photo-bioreactor for a photosynthetic culture having an aqueous liquid. The sun-tracking light distributor system comprises two light distribution walls, provided at an angle and oriented to converge at a bottom end, the light distribution walls being made of a transparent material allowing sunlight to pass therethrough, the two converging light distribution walls creating an elongated V-shaped channel with an interior space adapted to receive the sunlight and an exterior surface adapted to be partly immersed in the aqueous liquid in use; and a displacement system operatively connected to at least one of the two light distribution walls, the displacement system being adapted to change an orientation of at least one of the two light distribution walls to track a solar position with respect to at least one axis.

According to another broad aspect of the present invention, there is provided a method for distributing light in one of an open-ended photo-bioreactor and a closed photo-bioreactor for a photosynthetic culture, comprising: providing a sun-tracking light distributor system in one of an open-ended photo-bioreactor and a closed photo-bioreactor; and changing an orientation of at least one of the light distribution walls using the displacement system to allow tracking of the sun.

In the present specification, the term “transparent” is intended to mean a material which allows sunlight of the wavelength band of interest to pass therethrough with limited absorption in the material itself. For the production of algae, an example wavelength band of interest can be visible light in the range of 400 nm-700 nm.

### BRIEF DESCRIPTION OF THE DRAWINGS

Having thus generally described the nature of the invention, reference will now be made to the accompanying drawings, showing by way of illustration example embodiments thereof and in which:

FIG. 1 includes FIG. 1A, FIG. 1B and FIG. 1C in which FIG. 1A shows the position of the sun in the sky within daytime for locations close to the 45^{th} parallel of northern latitude, in the summer, FIG. 1B in the fall and spring and FIG. 1C in the winter;

FIG. 2 includes FIG. 2A and FIG. 2B in which FIG. 2A shows open-ended transparent V-shaped structures in perspective in an open-ended basin or pond and FIG. 2B shows a side view of the open-ended transparent V-shaped structures of FIG. 2A;

FIG. 3 shows closed transparent V-shaped structures in an open-ended basin or pond with the reflections of the light rays being apparent;

FIG. 4 includes FIG. 4A to 4C in which FIG. 4A shows an elongated tube or conduit for the transport of aqueous liquid, FIG. 4B shows a side view of the system with a plurality of adjacent rows of tubes of the type of FIG. 4A and FIG. 4C shows a perspective view of the rows of elongated tubes without the support system to show an example positioning of the tubes on the ground;

FIG. 5 includes FIG. 5A and FIG. 5B in which FIG. 5A and FIG. 5B show an example orientation of the V-shaped structures in an open-ended system for the summer months and the fall months, respectively, in a moderate climate region;

FIG. 6 includes FIG. 6A and FIG. 6B in which FIG. 6A shows an example Fresnel lens and FIG. 6B shows the example Fresnel lens at the light entry surface of a closed V-shaped light distributor;

FIG. 7 includes FIG. 7A and FIG. 7B in which FIG. 7A shows a hollowed triangular structure manufactured to include a water chamber and FIG. 7B shows a hollowed triangular structure with a plurality of chambers or cavities formed therein;

FIG. 8 shows a V-shaped structure with a perforated channel for transport and distribution of gas;

FIG. 9 includes FIG. 9A and FIG. 9B in which FIGS. 9A and 9B show an embodiment of the V-shaped structure with an inclined end wall, the V-shaped structure adapted to be lifted at the opposite end to allow disposal of waste and water at the inclined wall;

FIG. 10 shows a V-shaped structure with a perforated channel at its bottom for transport and distribution of gas, a dry air channel and a channel for a transparent liquid;

FIG. 11 shows a mirror affixed to the top of the exterior wall of the V-shaped structure;

FIG. 12 includes FIG. 12A and FIG. 12B in which FIG. 12A shows the penetration of light in a basin without light distributors and FIG. 12B with V-shaped light distributors;

FIG. 13 includes FIG. 13A and FIG. 13B in which FIG. 13A shows and an example displacement system which orients the light distributors using a handle for Summer and FIG. 13B for Fall;

FIG. 14 shows a gear arrangement to orient the light distribution walls;

FIG. 15 shows a variant displacement system in which the light distributors are made to rotate via an individual gear which is rotated by dented rods;

FIG. 16 includes FIG. 16A to 16E which show different methods to displace the aqueous liquid in the basin and therefore to displace the algae from one end of the basin to the other;

FIG. 17 shows a basin structure with multiple basins of varying sizes;

FIG. 18 includes FIG. 18A and FIG. 18B in which FIGS. 18A, 18B show the effect of the displacement system on the shape and angle of the tubing;

FIG. 19 includes FIG. 19A and FIG. 19B in which FIGS. 19A, 19B show an example tubing provided with internal channels and a holding structure;

FIG. 20 shows an example tubing with a mechanical structure to give a shape to the tubing;

FIG. 21 includes FIG. 21A and FIG. 21B in which an example tubing with a pressurized air/water channel which controls the shape of the algae channel is shown with a high pressure channel in FIG. 21A and a low pressure channel in FIG. 21B;

FIG. 22 shows a mirror affixed to the top of the attachment structure;

FIG. 23 shows a displacement system for the elongated tube;

FIG. 24 shows a weighted rigid V-shaped structure provided in an open-ended basin with a sheet of thin film maintained at the interface between the rigid V-shaped structure and the aqueous liquid;

FIG. 25 shows a perspective view of the V-shaped structure of FIG. 24;

FIG. 26 shows an armature with transparent walls to create V-shaped surfaces;

FIG. 27 shows a perspective view of the armature of FIG. 26;

FIG. 28 shows an alternative to FIG. 26 in which a transversal rod is provided;

FIG. 29 shows the assembly of FIG. 26 with a thin film applied to the exterior wall;

FIG. 30 shows the assembly of FIG. 28 with a thin film applied to the exterior wall;

FIG. 31 shows an example V-shaped structure;

FIG. 32 shows the reflections of the sun rays for the V-shaped structures of FIG. 31;

FIG. 33 shows example V-shaped structures of FIG. 31 oriented to track the sun at the Spring and Fall equinoxes;

FIG. 34 shows the V-shaped structures of FIG. 31 oriented to track the sun in the summer months;

FIG. 35 shows an example V-shaped structure with an internal triangular structure;

FIG. 36 shows the example V-shaped structure of FIG. 35 oriented to track the sun at the Spring and Fall equinoxes; and

FIG. 37 shows the example V-shaped structure of FIG. 35 oriented to track the sun in the summer months.

It will be noted that throughout the appended drawings, like features are identified by like reference numerals.

### DETAILED DESCRIPTION

In order to allow efficient light distribution in open and closed photosynthetic culture aqueous systems which use the sun as a light source, a sun-tracking light distributor can be provided. The sun-tracking light distributor has a V-shaped light distribution surface and a light entry surface adapted to be oriented to track the sun. The light distributor is used to capture the light from the sun and distribute it within the volume of aqueous liquid. This light distributor forces some light to travel from above the surface of the aqueous liquid to a depth within the volume of aqueous liquid. The light distributor is to be oriented so as to be able to capture a significant portion of the light from the sun and distribute it within the volume of aqueous liquid, regardless of the time of day or day of year.

The photosynthetic culture may comprise algae and other species that use photosynthesis, such as microorganisms. Photosynthetic culture is known to require a light intensity much lower than the maximum solar light intensity. Studies have shown that optimal intensities for photosynthesis can be of the order of 10% of the maximum solar light intensity. Light distribution within an aqueous system is often referred to as “light dilution” in the field of algae culture. The higher the light dilution factor, the more significant the impact on the production of algae.

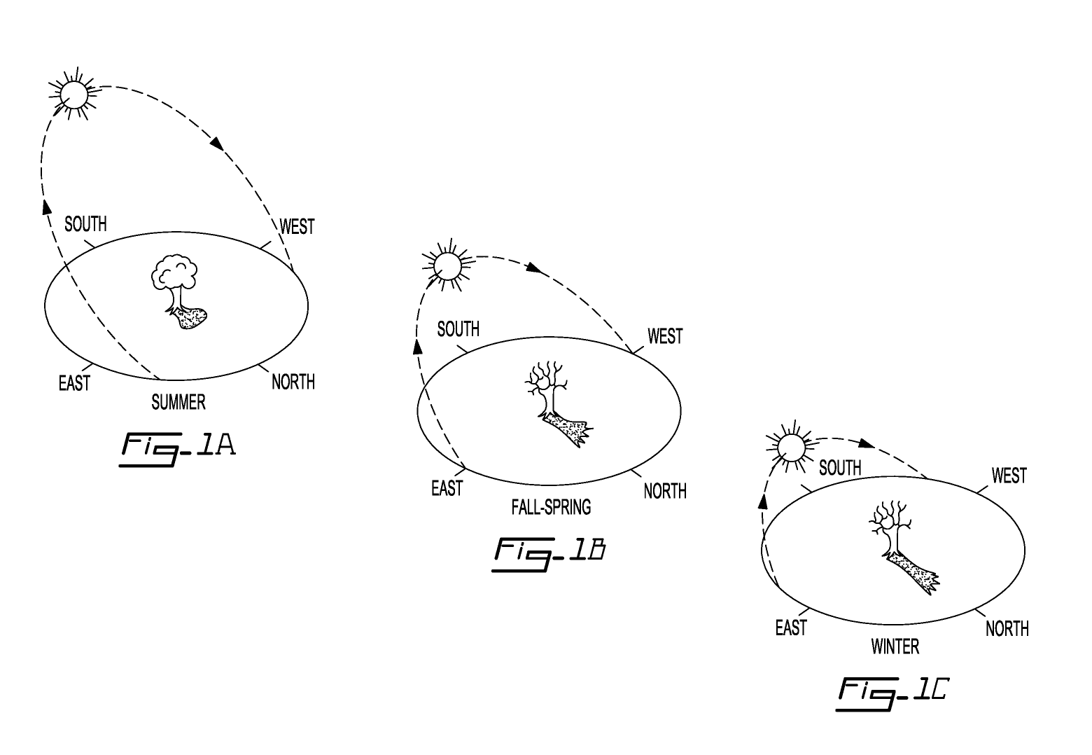

It is well known that the sun moves westward in the sky during daytime and that its maximum altitude (culmination) changes throughout the year. The sun is at its maximum altitude at solar noon each day. Solar noon occurs exactly half way between sunrise and sunset. In the northern hemisphere, the sun is due south at solar noon. The sun altitude passes from about 68° at summer solstice to about 21° at winter solstice for locations close to the 45^{th} parallel of latitude. At these latitudes the maximum solar altitude at both spring and fall equinoxes is about 45°. FIG. 1 illustrates schematically the sun path for a location close to the 45^{th} parallel of latitude. In FIG. 1A, the sun trajectory is shown for a summer day, in FIG. 1B, for a fall or spring day and in FIG. 1C, for a winter day.

In use in a photosynthetic culture aqueous system, a plurality of adjacent V-shaped light distribution surfaces will be provided at the interface between the air and the aqueous liquid. The adjacent V-shaped light distribution surfaces have a longitudinal dimension much greater than the height of their V-shape, thereby creating elongated V-shaped channels.

At least one of the light distribution wall of the V-shaped surfaces will be adapted to be inclined as a function of the maximum solar angle. The longitudinal axis of these V-shaped channels can be oriented along the East-West direction. Alternatively, the longitudinal direction of the elongated V-shaped channel can be disposed in a North-South orientation. The displacement system will typically need to adjust the inclination of the light distribution wall(s) less frequently if the longitudinal direction of the elongated V-shaped channel is in the East-West direction instead of the North-South direction. As will be readily understood, one can choose to dispose the longitudinal axis of the elongated V-shaped channels in an orientation other than North-South or East-West and still adjust the inclination of at least one of the two light distribution walls using the displacement system in response to the solar inclination throughout the day and the year.

In order to achieve a light dilution factor of 5 to 10, each face of the V-shape light distribution surface can be at an angle of about 5 to 15 degrees with respect to the orientation of the sun rays. The dilution factor is most difficult to achieve at the moment when the sun is at its highest point. The inclination of the V-shaped surfaces can therefore be calculated and positioned using the maximum altitude. For the rest of the day, the sun rays will be sufficiently well oriented to still be captured within the light distributors and distributed within the volume while yielding light dilution factors higher than at the time of the maximum sun angle. In most situations, the orientation of the V-shaped surfaces will therefore not need to be changed during the day and can only be modified once a day or even once every few days, in order to maintain an efficient light dilution factor at the time of the maximum sun angle. As will be readily understood, should one wish to adjust the orientation more often, it will be directly possible to do so. Any frequency of adjustment of the inclination of the V-shaped surfaces may be used. As will be readily understood, the tracking may be approximate, within a precision range acceptable for the application.

The surface area of the light distribution surface may largely exceed that of the light entry surface. For example, the light distribution surface may be 3 times the size of the light entry surface. More particularly, it may be 5 to 10 times the size of the light entry surface.

The light distribution surface allows penetration of light within the volume of aqueous liquid at a depth greater than the natural depth of light penetration in the volume of aqueous liquid. For example, the depth of penetration of light in the volume where light distributors are used can be 3 or even 5 to 10 times the natural depth of light penetration in the same aqueous liquid.

The sun-tracking light distributor system for a photosynthetic culture in aqueous liquid includes two light distribution walls provided at an angle and oriented to converge at a bottom end to create a V-shape. The light distribution walls are made of a transparent material allowing sunlight to pass therethrough. An interior side of the two light distribution walls creates an elongated V-shaped channel. The sunlight is received in the elongated V-shaped channel and is distributed through the light distribution walls in the aqueous liquid. The light distribution walls are disposed in the photosynthetic culture such that at least part of an exterior side of the walls is in contact with the aqueous liquid. The system also includes a displacement system adapted to change an angle of at least one of the two light distribution walls in response to the solar inclination.

Different example embodiments are proposed for the open-ended systems and the closed systems.

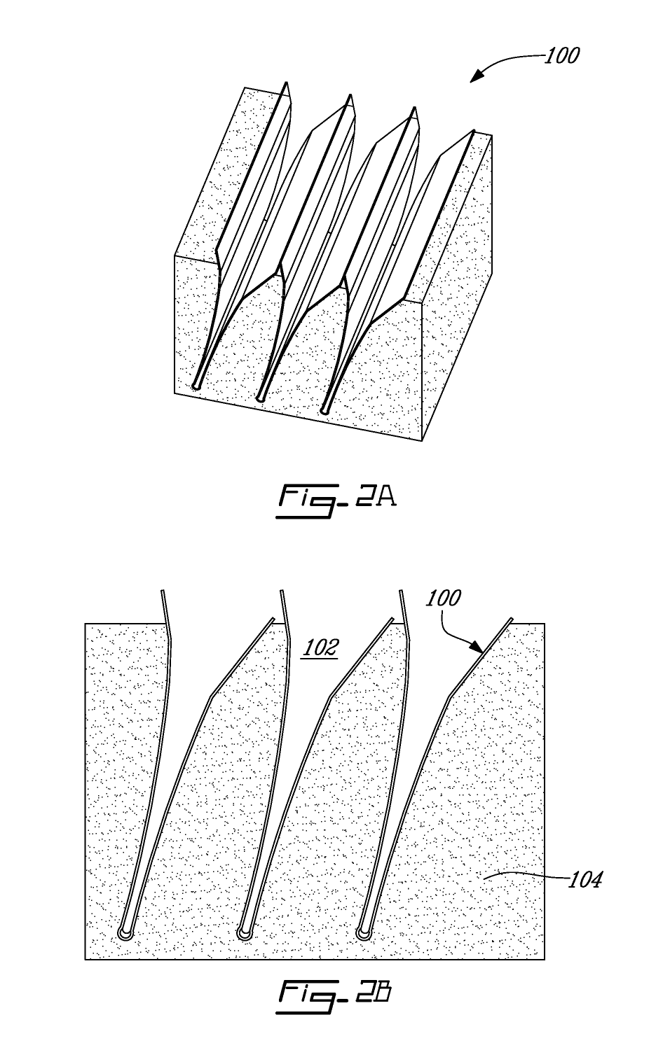

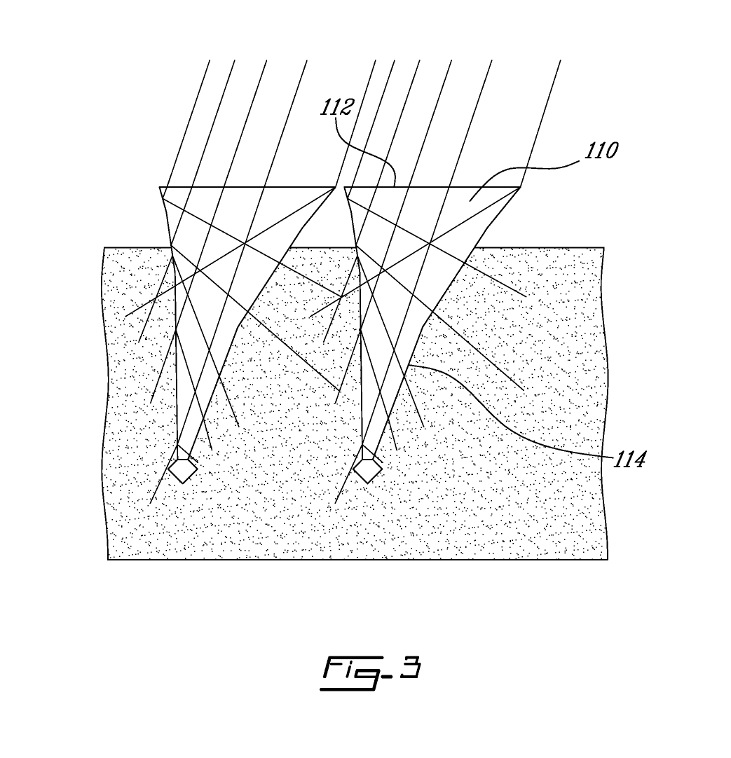

The transparent V-shaped structures are introduced at the surface of the open-ended systems. With reference to FIG. 2A and FIG. 2B, these transparent V-shaped structures can be open-ended 100. With reference to FIG. 3, these transparent V-shaped structures can be closed structures 110, with a substantially triangular shape, the triangle being inverted with its base at the top. The triangular V-shaped structures can be hollow and/or have channels formed therein.

When the photosynthetic culture is an open-ended system, the two light distribution walls are sidewalls of a V-shaped transparent structure and the two light distribution walls provided at an angle and oriented to converge at the bottom end are joined to form a unitary V-shaped transparent structure. A transparent top surface can be provided to close the V-shaped structure into an inverted-triangle structure.

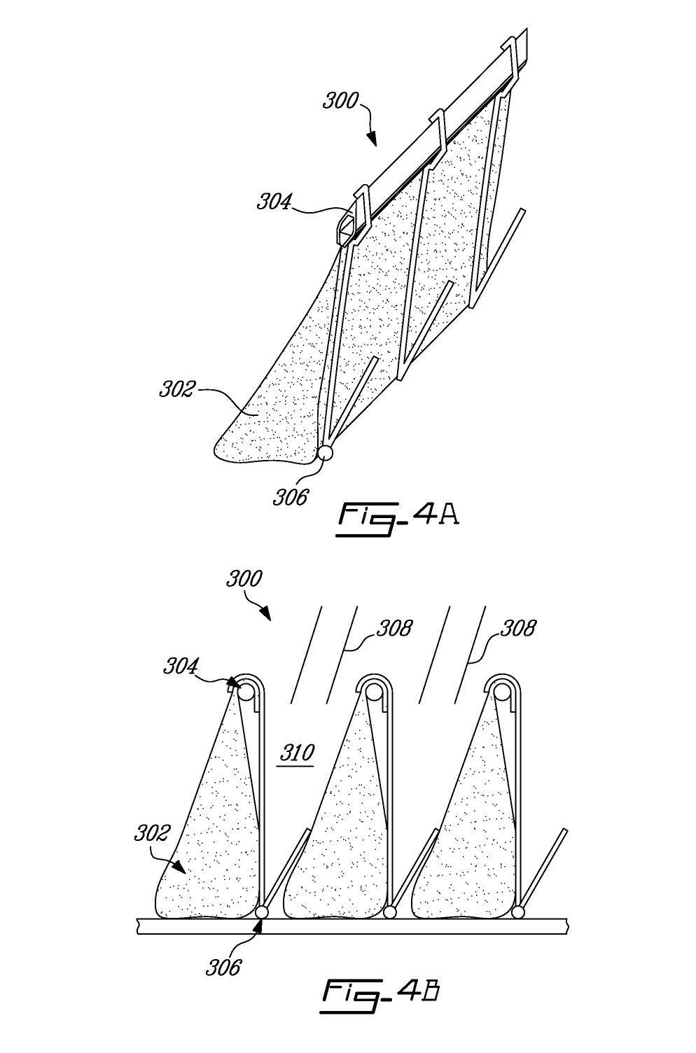

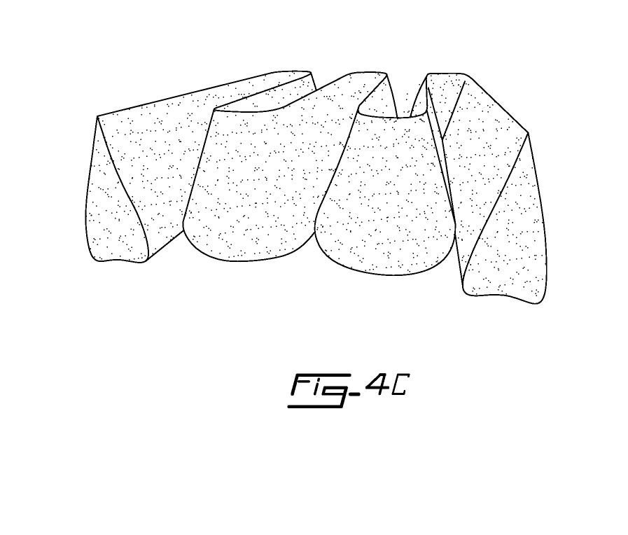

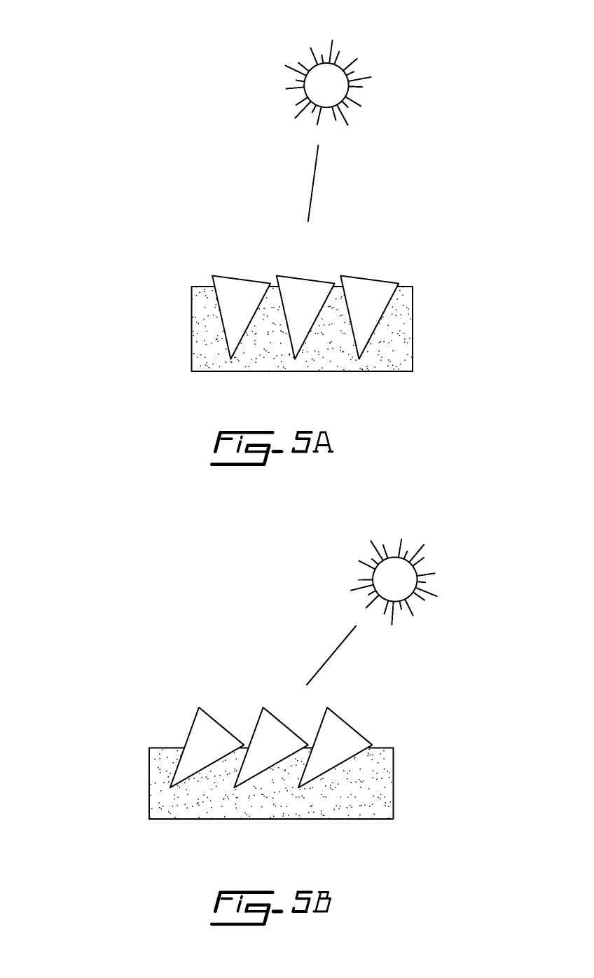

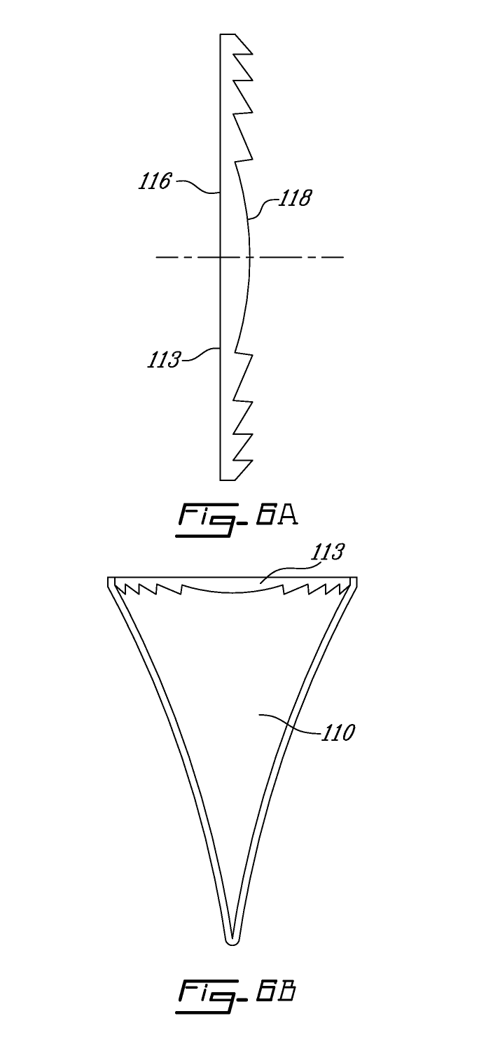

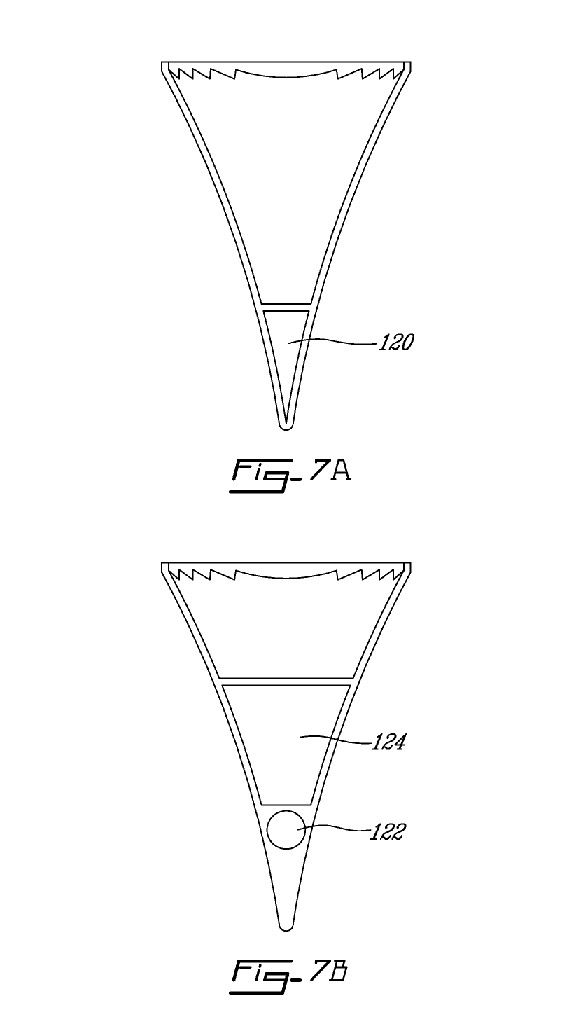

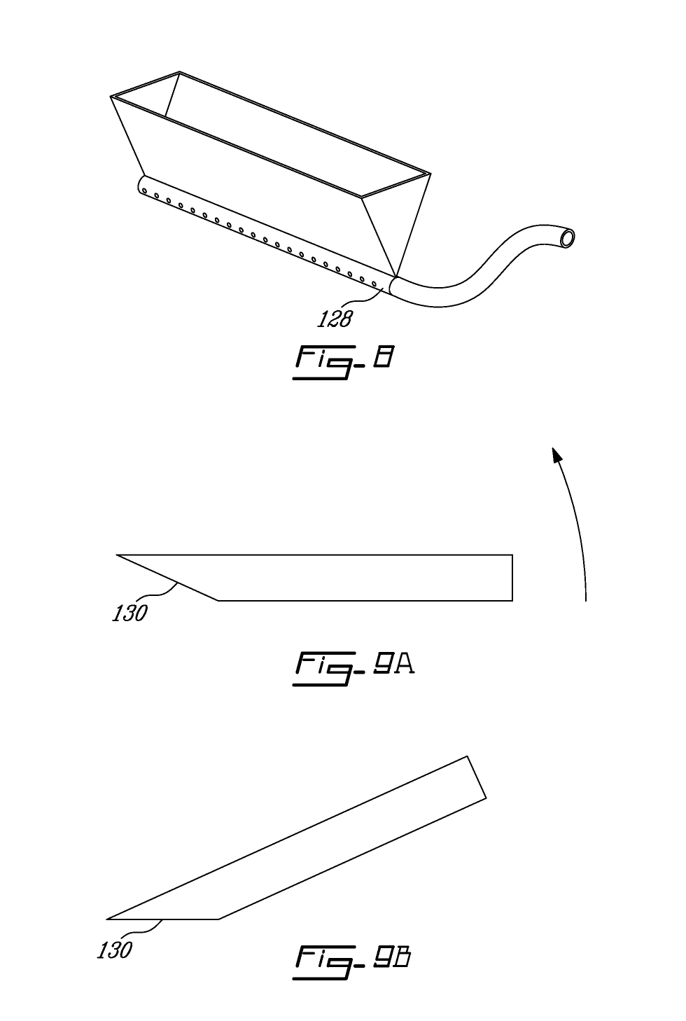

For closed systems, such as a PBR, elongated conduits 302 are shaped and juxtaposed to create a V-shaped region 310 between the rows of conduits 302. See FIGS. 4A, 4B and 4C. The elongated conduits are made of a thin transparent material and the aqueous algae culture occurs within the conduits. The elongated conduits are pulled from the top, creating substantially flat sides. The sides of the adjacent elongated conduits therefore act as the light distribution walls and the solar rays are able to be distributed within the conduits.

FIG. 5A and FIG. 5B show an example orientation of the V-shaped structures in an open-ended system for the summer months and the spring/fall months, respectively, in a moderate climate region. The longitudinal axis of the elongated V-shaped surfaces are oriented in the East-West direction, the sun-tracking is performed along a single axis.

When the photosynthetic culture is in a closed photo-bioreactor, the light distribution walls are sidewalls of an elongated tube. In one embodiment, the elongated tube can be disposed in a S-shape so as to create a plurality of rows of the elongated tube. In another embodiment, elongated tubes are positioned to be adjacent and to form rows of elongated tubes. Manifolds are provided to interconnect the tubes. The rows of elongated tubes need not be strictly parallel or straight. The V-shaped channel is created by adjacent light distribution walls of adjacent rows of the elongated tube. The elongated tube includes a bottom wall to contain the photosynthetic culture between exterior sides of the light distribution walls.

The V-shaped channel is closed if the adjacent light distribution walls converge and meet at the bottom end. The V-shaped channel is open if the light distribution walls do not touch at the bottom end but are oriented such that imaginary extensions of the adjacent light distribution walls converge to meet at the bottom end. The bottom wall and the light distribution walls can be made of a single sheet of transparent material.

As will be readily understood, if a flexible material is used for the elongated tubes, the V-shaped channel will most likely not have strictly straight light distribution walls and the bottom end of the V-shaped channel may have a varying width along the longitudinal direction of the elongated tube. The light distribution walls may be curved or may bear sections with different angles. The light distributors walls can be asymmetrical. The light distribution walls may have different lengths.

Referring back to FIG. 2A and FIG. 2B, the V-shaped structures are rigid and create elongated V-shaped channels 102 in the basin. These V-shaped channels 102 are filled with air. The rigid material is transparent and can be made of plastic, glass or any other transparent material. It is, for example, made of PMMA, polycarbonate or even glass. It can be made of extruded plastic material. It can sustain the pressure from the aqueous liquid of the basin 104. The elongated channels 102 form chambers which capture and trap the solar rays. Unfortunately, dirt, water and other contaminants can accumulate in the channels. As will be readily understood, the open-ended region of the V-shaped structures is to be provided above the surface level of the aqueous liquid in the basin.

The open-ended region of the V-shaped structure can be covered with a transparent film (not shown) which can be affixed permanently or temporarily, for example by adhesion, to the top of the exterior surfaces of the V-shaped structure. This transparent film can prevent algae, dirt and aqueous liquid from entering the open-ended V-shaped structure. This transparent film can be replaced periodically.

An example open-ended V-shaped light distributor has a shape similar to that shown in FIG. 2B. In this example embodiment, the open-ended light entry surface has a transverse width of 10-20 cm. The light distribution wall has a height of about 30-50 cm. The length of the light distribution walls will depend on the rigidity of the materials used and of the structure created.

Referring now to FIG. 3, the triangular structures can be hollow. The triangular structure can be filled partly or fully with dry air, ethanol, glycerol, water or can be a prism of transparent material. A liquid or solid material can be inserted therein to facilitate light propagation.

The top surface of the inverted-triangle structure, namely the light-entry surface 112, will reflect some of the solar illumination towards the sky. This will result in losses. With a hollow triangle made of glass or PMMA filled with air, there will be a loss of about 8% at noon and about 15% of the total collected energy in a day. With a transparent material, for example water, which can be used within a prism cavity in the top section of the triangle, the losses will be reduced.

An example closed V-shaped light distributor has a shape similar to that shown in FIG. 3. The light entry surface 112 has a transverse width of 10-20 cm. The light distribution wall 114 has a height of about 30-50 cm. The length of the light distribution wall will depend on the rigidity of the materials used and of the structure created.

The light entry surface 112 is provided above a surface of the aqueous liquid and the light distribution walls 114 are provided partly underneath the surface of the aqueous liquid.

The light entry surface 112 can be provided with a polymer material with a low refractive index to lower the reflection losses.

A removable transparent film can be applied on the exterior surfaces of the V-shaped structure. This transparent film can help in keeping the wall intact and may prevent scratches from being formed in the wall. Algae will accumulate on the film. The film can be removed and replaced when necessary.

The light entry surface 112 can bear a light concentrator element 113 which allows propagating light through a channel which has a cross-section smaller than the light entry surface. A Fresnel lens is an example of such an optical component. Other example light concentrator elements include a standard converging lens of suitable focal length, a mirror (metallic or dielectric) disposed on the side of the light distribution wall, etc. An example cylindrical Fresnel lens is shown in FIG. 6A. One of its surfaces is flat 116 while the other is ridged 118. The design of the Fresnel lens allows the fabrication of a lens of large aperture and short focal length, while requiring less material than a conventional lens of similar characteristics. It can therefore be used to redirect the solar rays within the channel. FIG. 6B shows a closed V-shaped light distributor 110 with a Fresnel lens 113.

To facilitate cleaning, the flat surface 116 of the Fresnel lens may be set external to the light distributor and the ridged surface 118 may be towards the interior of the light distributor to concentrate the solar rays towards the interior of the light distributor. According to a variant, the flat surface may be towards the interior of the light distributor and the ridged surface towards the exterior of the light distributor.

As shown in FIGS. 7A and 7B, the hollowed triangular structure can be manufactured to include a water chamber 120, 122 to assist, for example, in sinking of the V-shaped structure. A channel 124 for a transparent liquid, for example water with facultative additives can also be provided. As shown in FIG. 8, it can also be manufactured to include a perforated channel 128 for transport and distribution of air, CO₂ and/or gas within the aqueous system.

As shown in FIGS. 9A and 9B, one longitudinal end of the open-ended elongated V-shaped structure can have an inclined end wall 130 and the open-ended V-shaped structure can be adapted to be lifted at the opposite end to allow disposal of waste and water at the inclined end wall. This can be useful for cleaning purposes, for example.

As shown in FIG. 10, the V-shaped structure can be made of extruded plastic material and can be shaped to include a few useful features. For example, a perforated channel for transport and distribution of air and/or CO₂ can be provided at the bottom of the V-shaped structure 140. A dry air channel 142 having a shape adapted for floatation is also provided. Pressurized air could be provided in dry air channel 142 to help compensate for hydrostatic pressure. A channel for a transparent liquid 144, for example water with facultative additives can also be provided. Air cavities 146 can be created in the walls of the V-shaped structure to facilitate total internal reflection within the V-shaped structure.

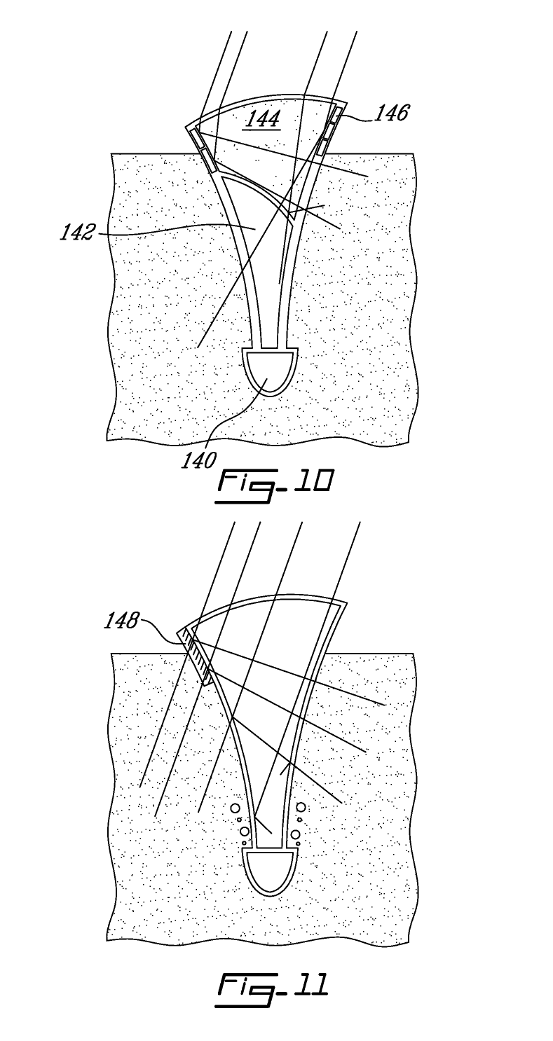

As shown in FIG. 11, a mirror 148 can be affixed or provided at the top of the exterior surface of the V-shaped structure to facilitate reflection within the V-shaped structure. The reflector can be a sheet of aluminum with a coating of silver which can extend above and/or below the surface of the aqueous liquid.

Multiple light distributors which may or may not be identically sized and shaped, can be provided in the aqueous system such that the light entry surface of adjacent light distributors covers most of the open-ended surface of the system.

As will be readily understood, at some latitudes and for some algae concentrations in the aqueous liquid, it may be preferable to have narrower V-shaped structures, for which the width of the light entry surface is reduced and the height of the light distribution walls from the light entry surface to the bottom of the V-shaped structure is increased.

Simulations were carried out to determine the difference in light penetration between an open-ended photo bioreactor without solar tracking light distributors and one with solar tracking light distributors. FIG. 12 shows the penetration of light for a V-shaped light distributor. In FIG. 12A, the results are shown for system without light distributors. In FIG. 12B, the system includes light distributors. As shown in FIG. 12A, most of the sunlight is absorbed in a thin layer at the top of the photo bioreactor and the light intensity in that layer is at least one order of magnitude above the optimal value for efficient algae production. FIG. 12B shows that the light distributors distribute the sunlight in a much larger volume of aqueous liquid and that most of the volume is illuminated with an intensity closer to the optimal value.

A displacement system allows changing the orientation of the light entry surface of the multiple light distributors in the photo-bioreactor to allow tracking of the position of the sun. The displacement system allows proper positioning of the light entry surface of each light distributor generally towards the sun, depending on its current position. This displacement system can be individual for each light distributor or can be a global displacement system which controls a plurality of light distributors or all of them. The displacement system displaces the light distributors along at least one axis.

An example displacement system is shown in FIGS. 13A (summer) and 13B (fall). In this example displacement system 200, each V-shaped structure 202 is attached to two elongated rods 204, 206. The rods 204, 206 are displaced by a handle 208 which is also attached to the two elongated rods 204, 206. The movement of the handle 208 displaces each rod 204, 206 in an opposite direction and therefore forces the V-shaped structures 202 to pivot. The movement of the handle 208 can be controlled by an actuator.

The handle can receive a manual input to change the orientation of the light distributors or the actuator can use sensors and a controller to detect the position of the sun and orient the light distributors automatically and accordingly. The controller can provide an actuation command for the actuator(s). The controller may use stored solar position data to prepare the actuation command. For example, tables including the solar position for the time of day and day of year can be used by the controller. The controller may receive a manual input from a user to prepare the actuation command. The actuator(s) may also directly receive a manual input from a user to displace the light distributors. Feedback signals can be used to adjust the position and/or the position can be preprogrammed according to sun inclination projection data.

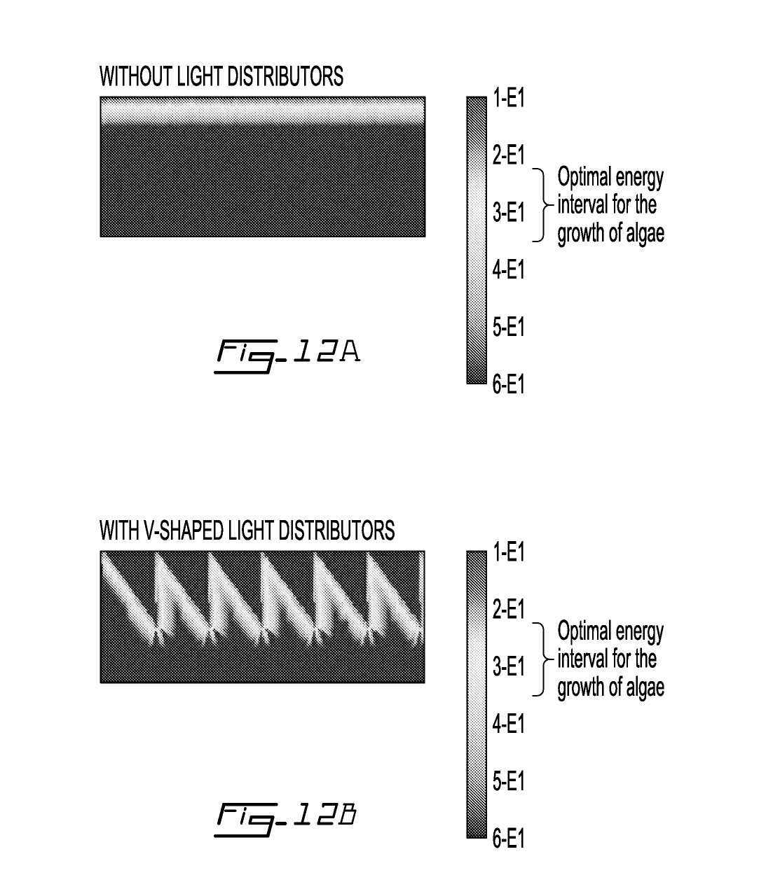

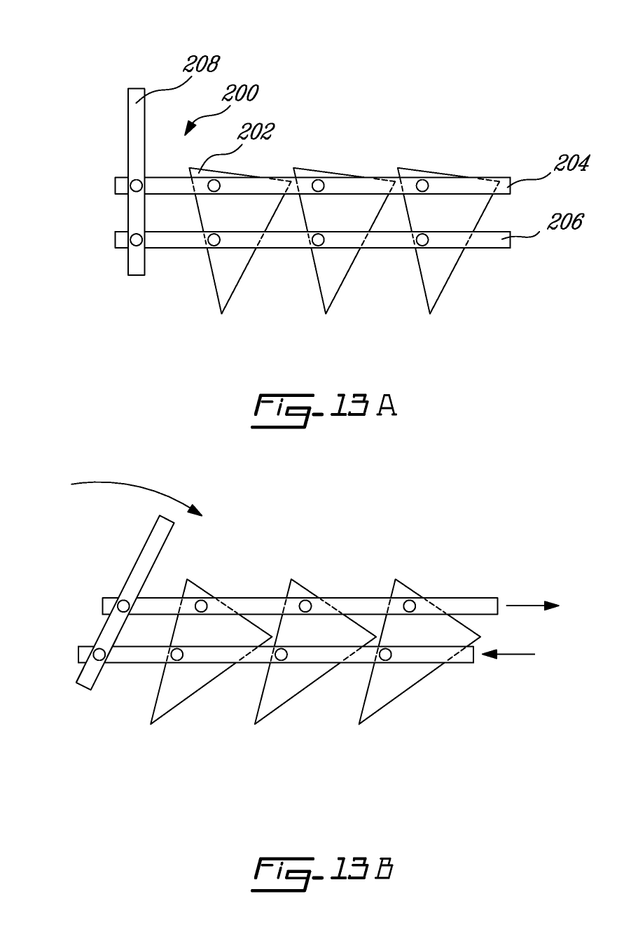

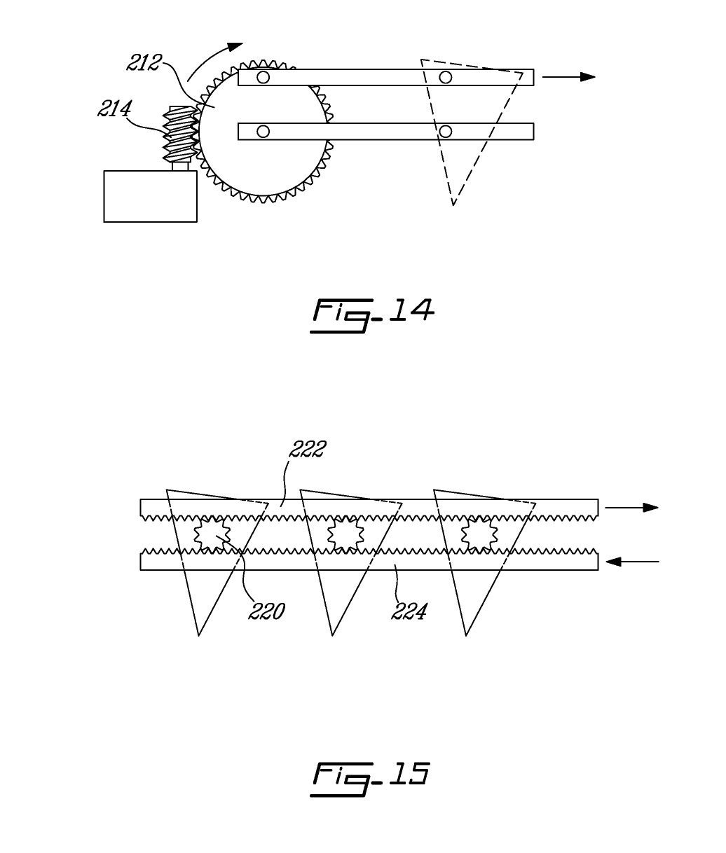

FIG. 14 shows a gear arrangement 212, 214 which is a variant to the handle of FIG. 13.

FIG. 15 shows a variant displacement system in which the light distributors are made to rotate via an individual gear 220 which is rotated by dented rods 222, 224.

Mechanical equivalents which allow to change the inclination of the light distribution walls will be known by the person skilled in the art.

As will be readily understood, the tracking may be approximate, within a precision range acceptable for the application. It is not necessary to fully track the position of the sun to benefit from light distribution improvement. The improvement in light penetration in the photo-bioreactor will be dependent on the actual percentage of light captured and distributed in the volume of aqueous liquid by the light distributors.

As will be understood, it may be determined that the photo-bioreactor algae production is inefficient in winter and is simply paused until spring.

It will also be understood that it may be advantageous to displace the algae to create a flow in the photo-bioreactor, allowing to continuously add water and nutrients after extracting some algae. It has been found that slow macro movements are preferable to rapid micro movements in order to avoid breaking the algae structure. Consequently, the photo-bioreactor may be shaped to allow a periodical displacement of the aqueous liquid with the algae and the light distributors may be installed in a manner facilitating this displacement and allowing the algae to travel, thereby benefiting from the light distribution of each light distributor which may, for any number of reasons, have varying light dilution factors. FIG. 16 shows different methods to displace the aqueous liquid in the basin and therefore to displace the algae from one end to the other using rotatable panels.

As will be readily understood, at the beginning of the production, the algae are in lesser concentration and the light penetration is high. Therefore, light distributors with large light entry surfaces in deep basins may be used. As the production progresses, light distributors with smaller light entry surfaces may be more appropriate and shallower basins may be used. At the end of the production, light distributors with small light entry surfaces are preferable in shallow basins. It may therefore be appropriate to create a basin structure which allows the algae to be transferred from one basin to the next according to its production status and in which the light distributor shape and basin size is adapted to the production status. This is shown schematically in FIG. 17.

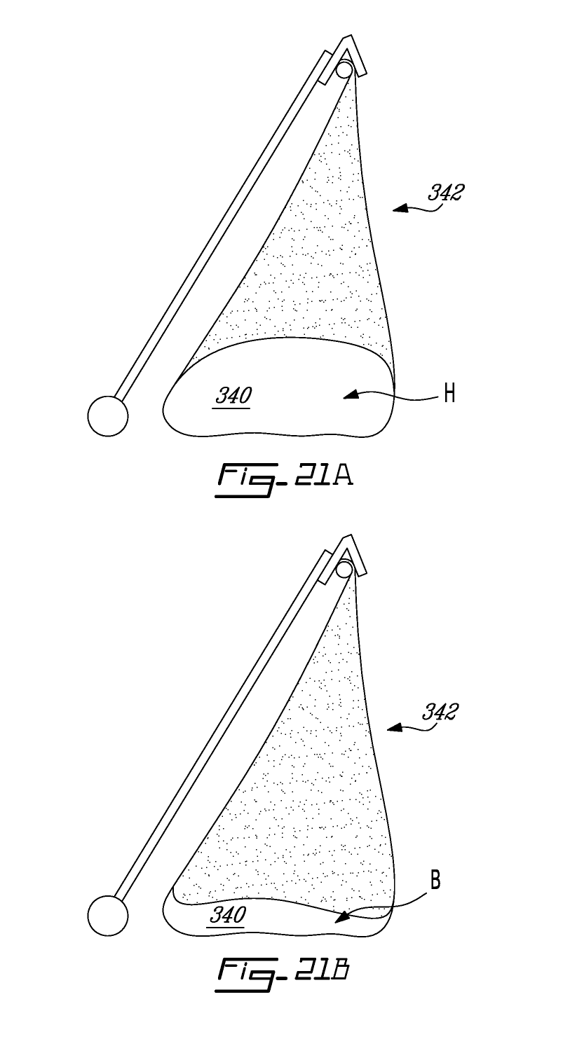

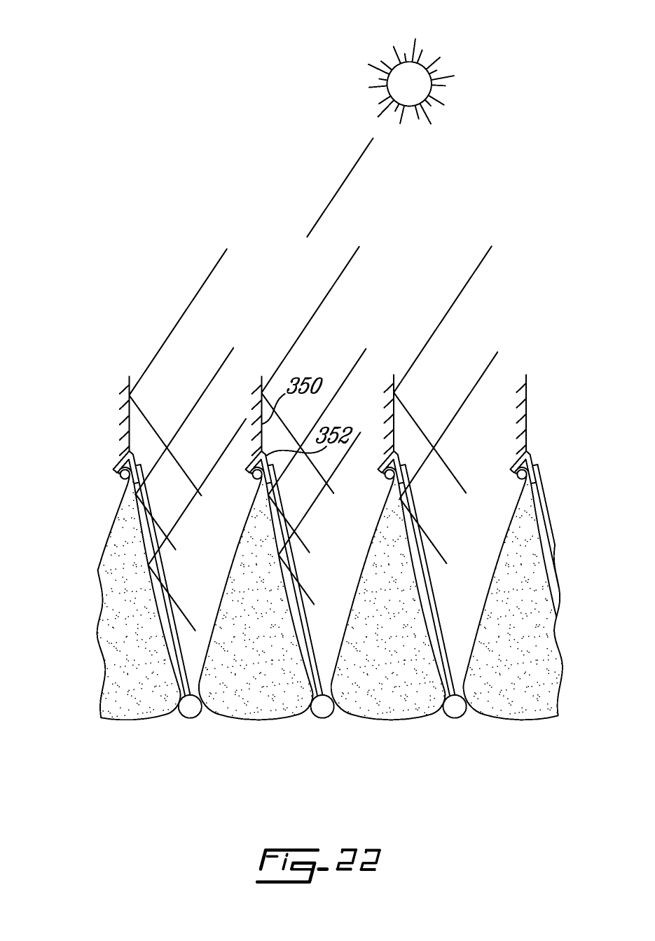

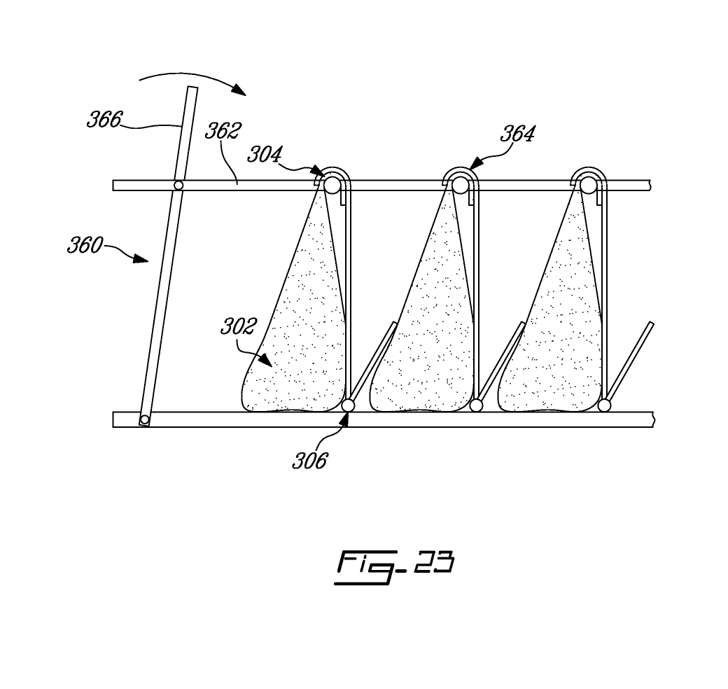

Another alternative embodiment for the light distributors to be used in open-ended basins is shown in FIGS. 24-25. As shown in FIG. 24, a weighted rigid V-shaped structure 230 is provided in an open-ended basin. This weighted rigid V-shaped structure can be made of metal or plastic. A sheet of thin film 232 is maintained at the interface between the rigid V-shaped structure 230 and the aqueous liquid 234 and is shaped by the structure to take on a V-shape. A rod 236 provided between adjacent rows of the V-shaped structure allows to maintain the film in between the rows. A reflector 238 can be provided on this rod 236 to redirect sun rays towards the V-shaped channel. FIG. 25 shows a perspective view of the V-shaped structure 230. The sheet of thin film 232 can be replaced periodically.

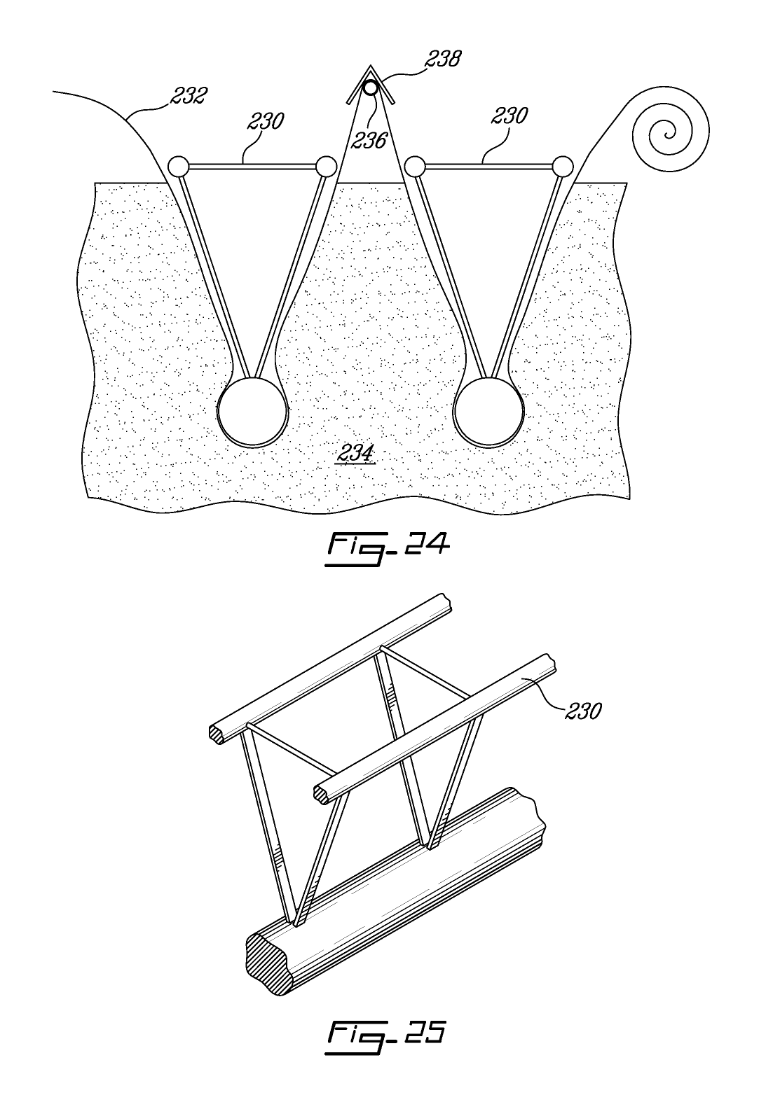

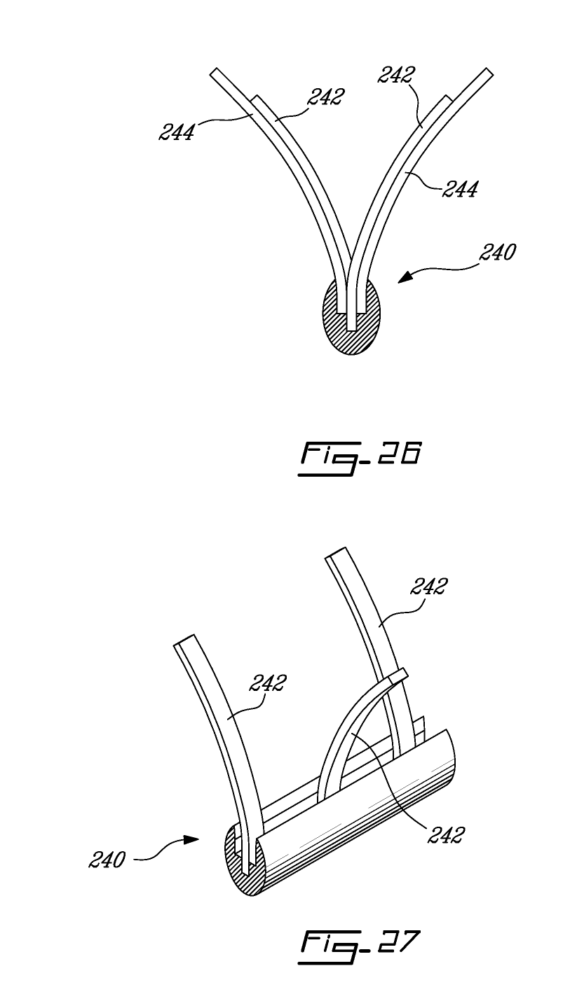

Still another alternative embodiment is shown in FIGS. 26-30. In this embodiment, a V-shaped structure 240 with a weighted base is provided in the basin. A strong armature 242 can give the V-shaped structure its shape and stability while transparent sheets of material 244, such as Poly(methyl methacrylate) (PMMA), are provided to create the V-shaped surfaces. FIG. 26 shows the assembled structure with the interior armature and the exterior layer of transparent material. FIG. 27 shows a perspective view of the armature. In FIG. 28, a transversal rod 246 is provided to solidify the structure. A thin film 248 can be applied to the exterior surface of the assembled V-shaped structure as shown in FIGS. 29 and 30.

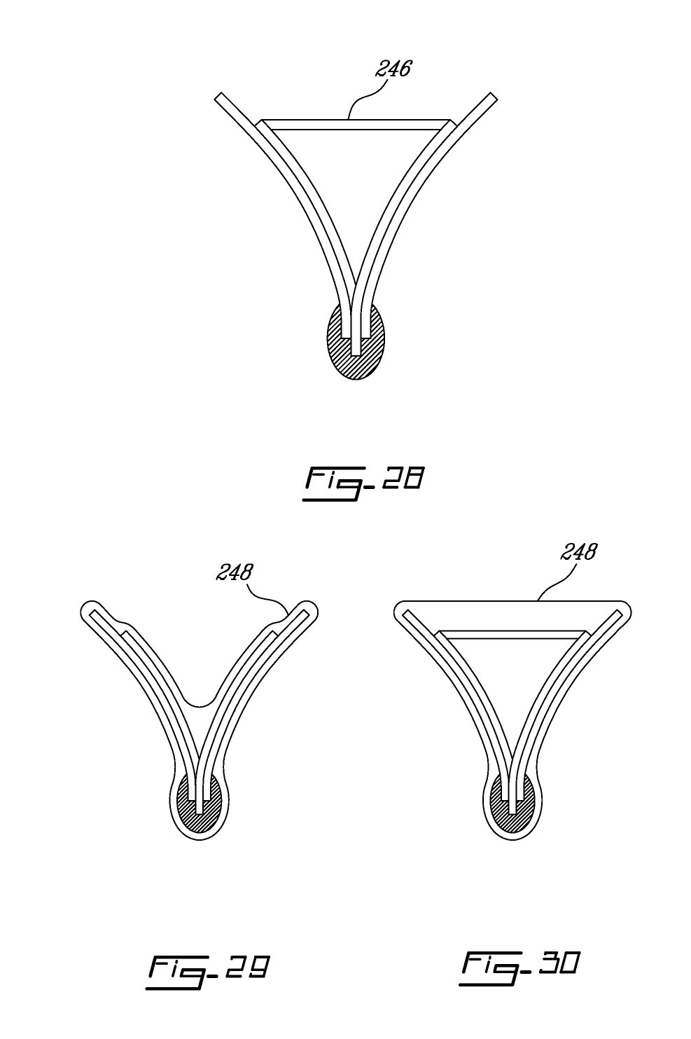

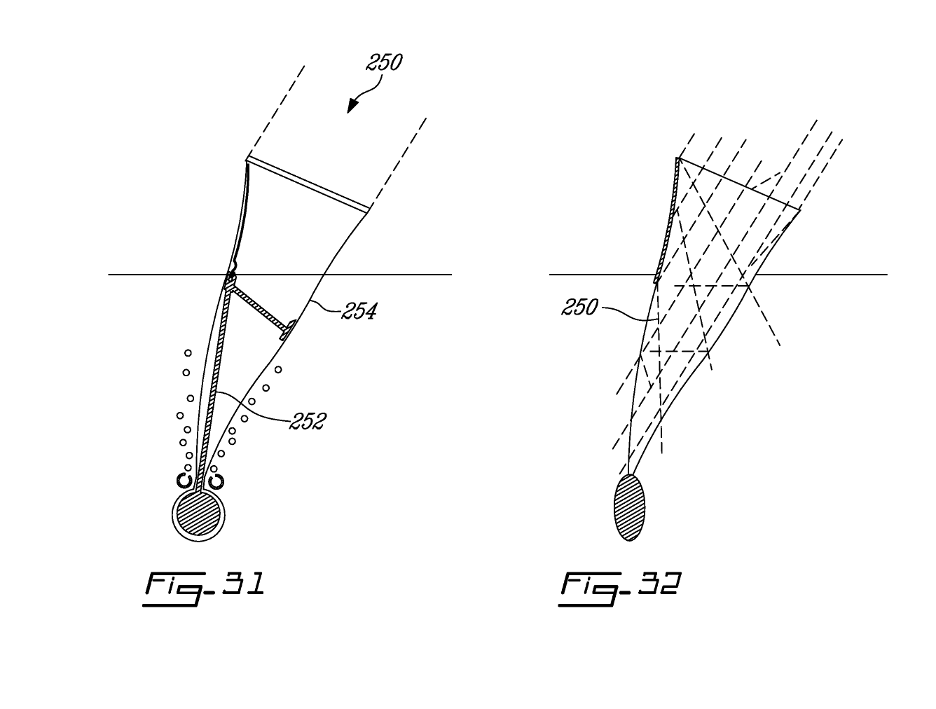

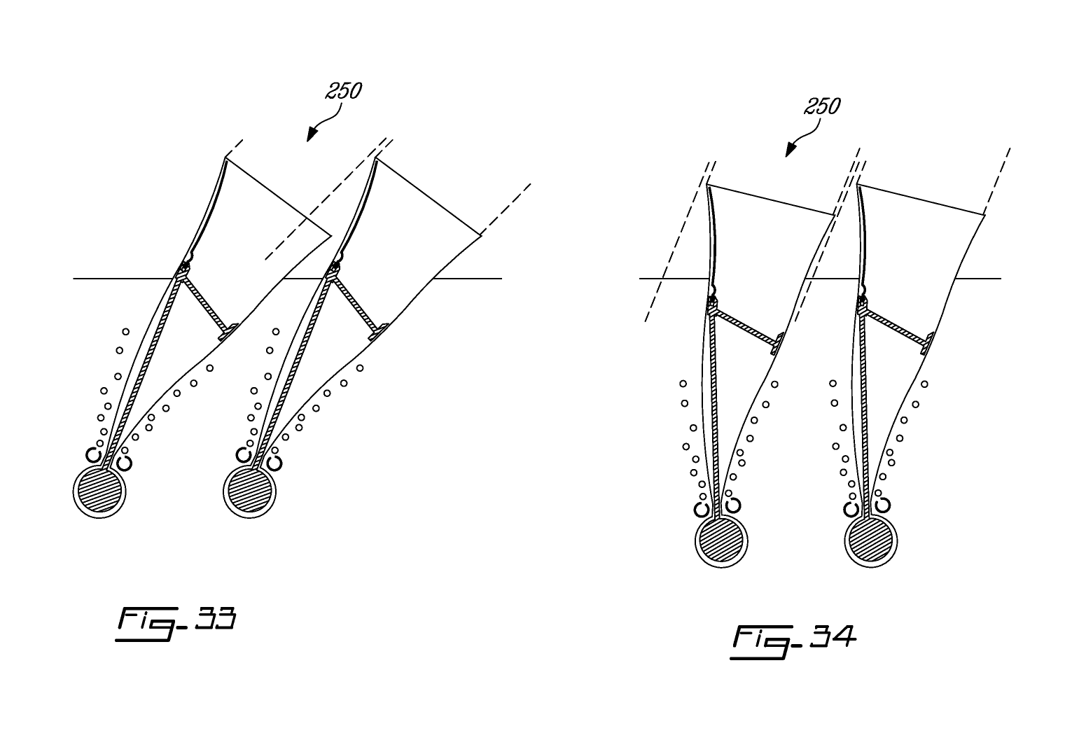

FIGS. 31 to 38 show other example embodiments for the light distributors to be used in open-ended basins. FIG. 31 shows an example V-shaped structure 250. The V-shaped structure includes an armature 252 and a layer of transparent material 254. FIG. 32 shows the reflections of the sun rays for an example V-shaped structure. FIG. 33 shows example V-shaped structures of FIG. 31 oriented to track the sun at the Spring and Fall equinoxes and

FIG. 34 shows the V-shaped structures of FIG. 31 oriented to track the sun in the summer months. In FIG. 31, the light entry surface has a width of 21 cm while the light distribution walls extend at least 10 cm above the surface of the aqueous liquid. The light distribution wall extends 35 cm below the surface of the aqueous liquid. There is a distance of 2 cm between adjacent V-shaped structures.

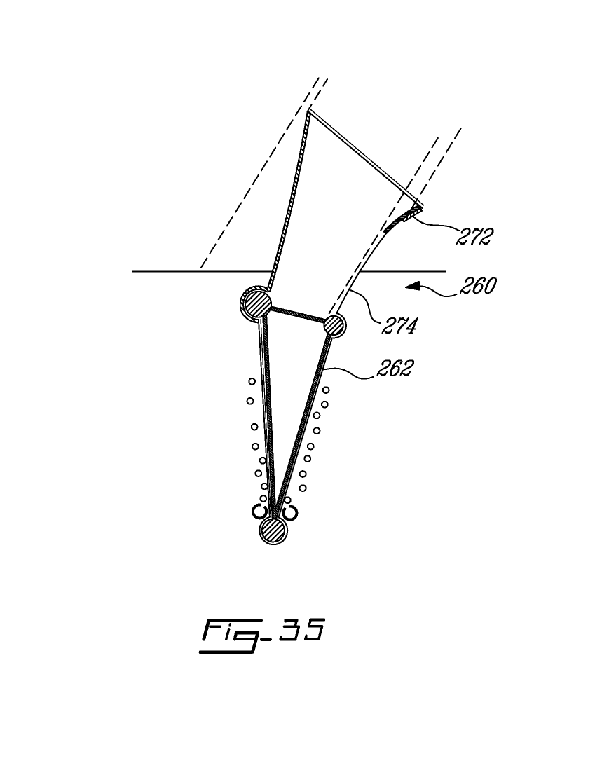

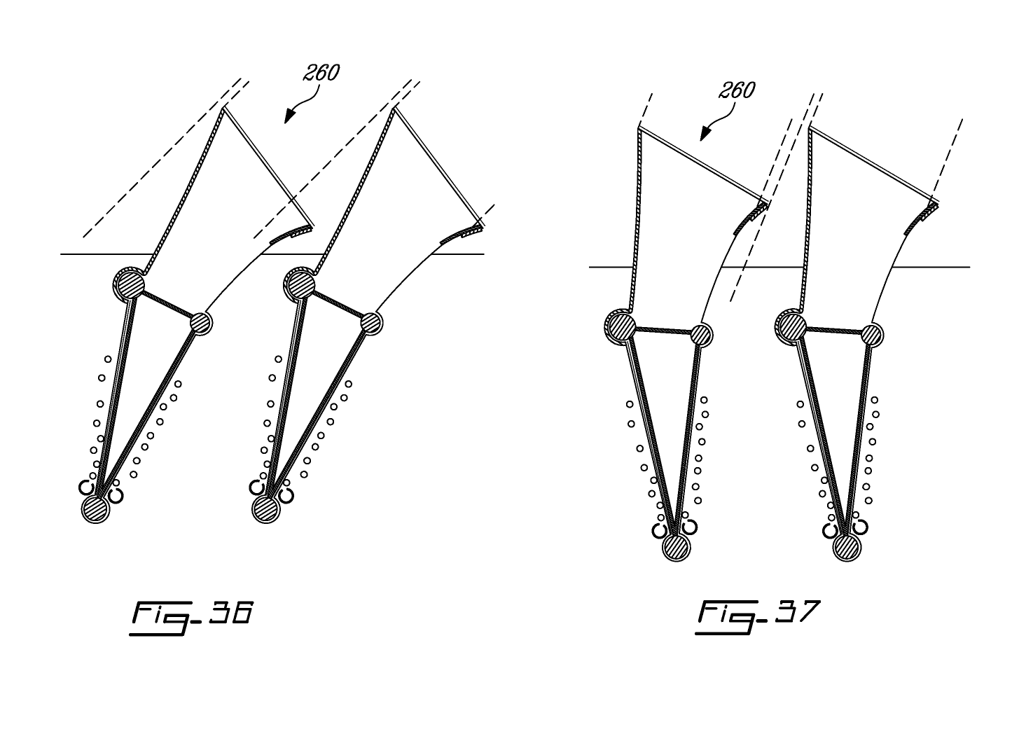

FIG. 35 shows an example V-shaped structure 260 with an internal triangular structure 262. An additional mirror 272, for example, made of a sheet of aluminum which can be coated with silver, is provided on the top of the most inclined light distribution wall 274 of the V-shaped structure 260. FIG. 36 shows the example V-shaped structure of FIG. 35 oriented to track the sun at the Spring and Fall equinoxes. FIG. 37 shows the example V-shaped structure of FIG. 35 oriented to track the sun in the summer months. In FIG. 35, the top of the V-shaped structure is provided at 26 cm above the surface of the aqueous liquid. There is a distance of 4 cm between adjacent V-shaped structures.

Referring back to FIG. 4A, FIG. 4B and FIG. 4C, for closed systems, such as a PBR, elongated conduits or tubes are shaped and juxtaposed to create a V-shaped region between the rows of conduits. The elongated conduits are made of a thin transparent material and the aqueous algae culture occurs within the conduits. The elongated conduits are pulled from the top. The sides of the adjacent elongated conduits therefore act as the light distribution walls and the solar rays are able to be distributed within the conduits. The walls of the tubing are the interface between the outside environment and the volume of aqueous liquid. The tubing walls can be oriented such that they follow the solar inclination throughout the year.

It will be readily understood that the resulting V-shaped structures between the rows of conduits have optical properties which are similar to that of the V-shaped structure of FIG. 2, namely the open-ended V-shaped structure.

In an example embodiment shown in FIG. 4B, the light distribution tubing system 300 includes an elongated tube 302, mechanical attachment means 304 and a displacement system with pivot 306 for participation in adjusting an angle of the tube depending on the solar inclination.

The elongated tube 302 is made of at least one sidewall. In one embodiment, it is made of a flexible thin material. The flexible material can be a polymer pellicle made up at least one layer. For example, thin plastic sheets can be used to form the tubing, similar to those used in the manufacturing of milk bags. Layers of these thin plastic sheets can be stacked with different optical, physical or biological properties to provide an ideal environment for the culture of the vegetal species. For example, in tubing made of three layers of polymers, an external layer can be UV-resistant or even UV-filtering, a middle layer can be significantly mechanically resistant and the internal layer can have a low adherence to the components present in the aqueous liquid.

In another embodiment, the elongated tube can be made of a rigid material. It can be an extruded polymer tube, for example.

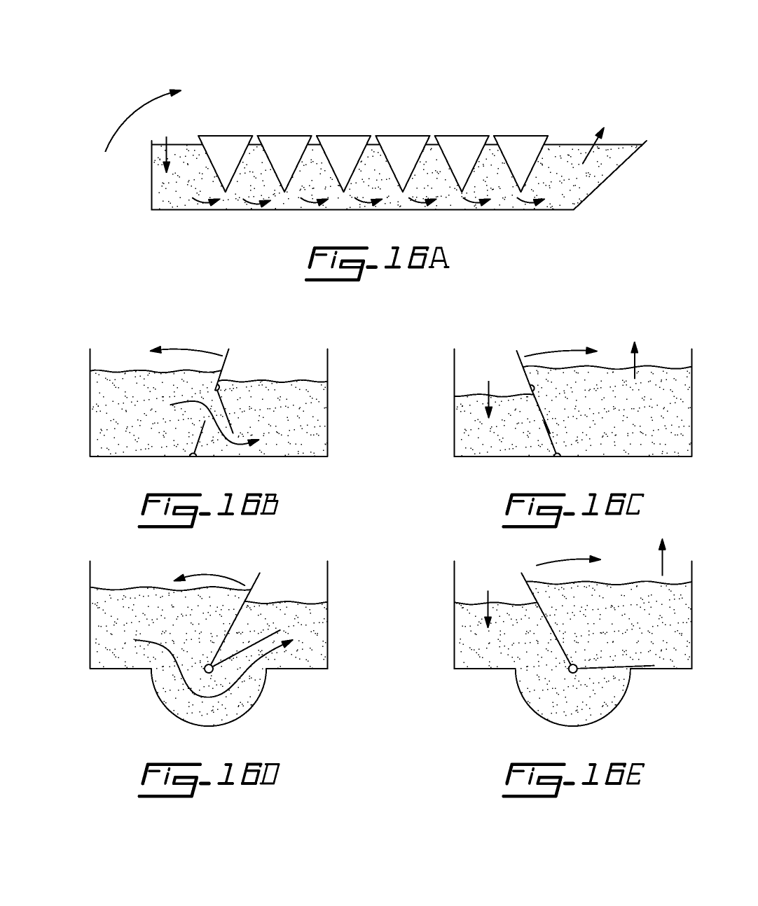

FIGS. 18A, 18B show the effect of the displacement system on the shape and angle of the tubing 302. The angle of both walls changes with the displacement of the attachment means and the wall is thereby adapted to capture sunlight 308 coming from different angles.

In an example tubing provided with internal channels, such as tubing 310 shown in FIGS. 19A, 19B, one channel 312 can be used to transport the plant and aqueous liquid, for example the algae in water, one channel 314 can transport carbon dioxide and distribute it to the algae channel along the way, one channel 316 can transport clear water to help shape the tubing in an appropriate shape or to create thermal communication with the ground surface, one channel 318 can transport air to create insulation or again to help with the shape of the tubing, etc.

The mechanical attachment means 320 attach the elongated top surface of the elongated tube to a support 322. The attachment means exert an upward force on the elongated tube to give it an upward pointing shape, a triangular shape, which pulls on the sidewall to give an angle to both sides, this angle being adapted for solar tracking. Furthermore, this triangular shape allows capturing the light and keeping most of it within the tube. The support 322 can include vertical rods 324 and a pivot 326.

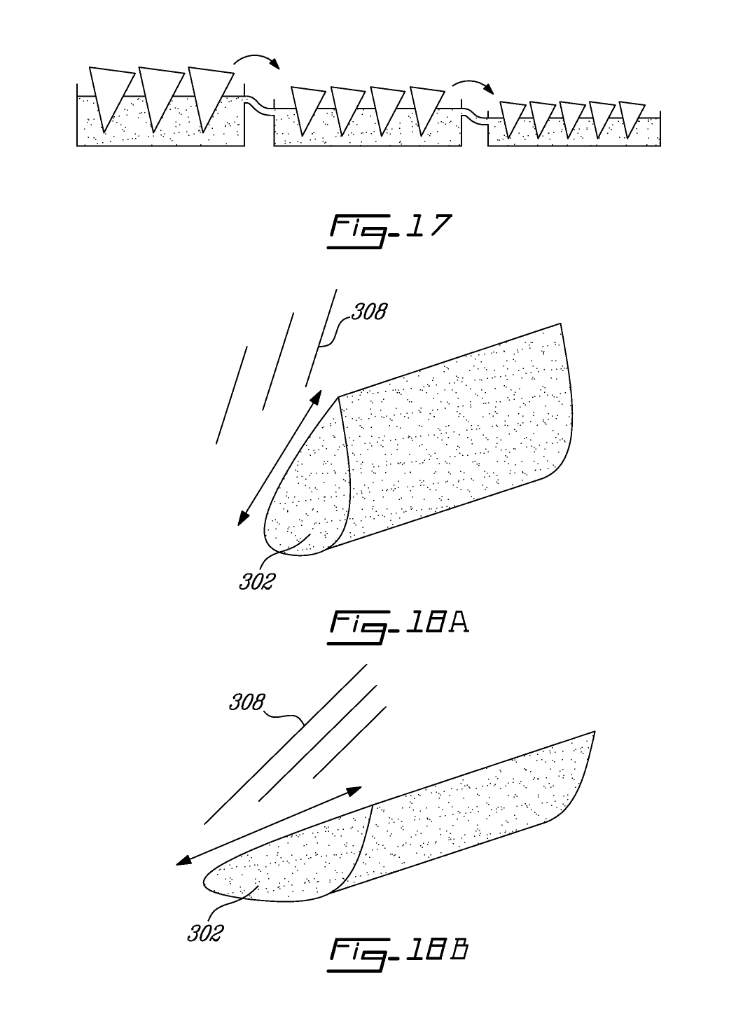

As shown in FIG. 20, a mechanical structure 330, having a triangular shape in this case, can be used to give a shape to the tubing.

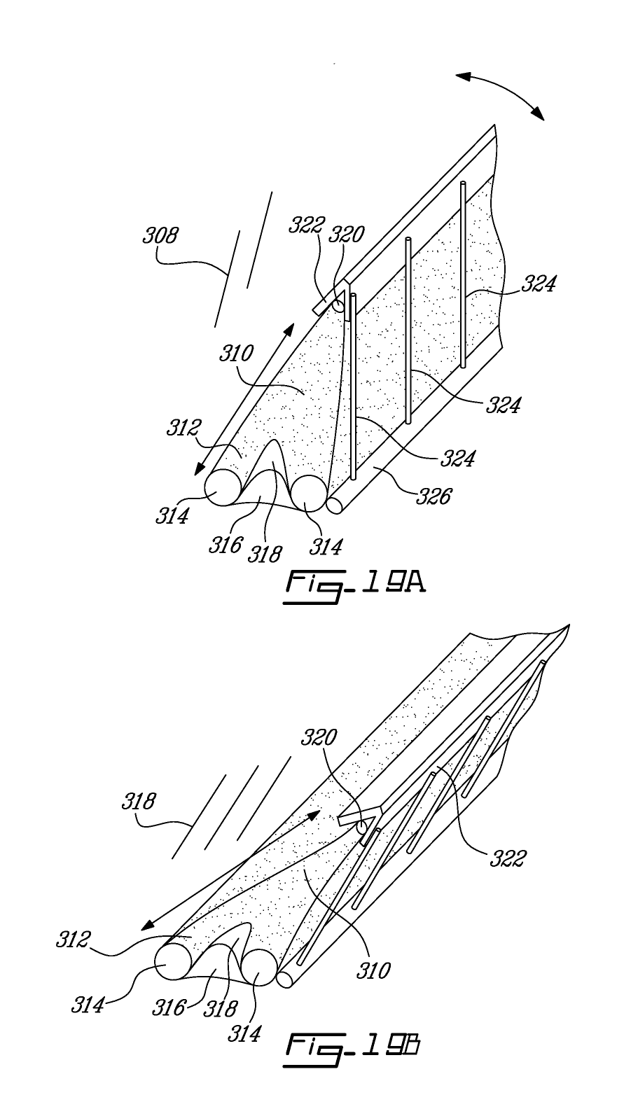

As shown in FIG. 21, the channels created in the elongated tube can allow regulating the quantity of aqueous liquid in the tubing. This can be useful to allow a lesser quantity of algae to circulate in the channel when the sun is at a lower angle. A pressurized air or water channel 340 is provided at the bottom of the tubing 342 and, as shown in FIG. 21A, if the pressurized air or water is at a high pressure (H), it will reduce the size of the aqueous liquid channel. As shown in FIG. 21B, if the pressurized air or water channel is at low pressure (B) it will increase the size of the aqueous liquid channel.

In another embodiment, not shown, the aqueous liquid can circulate in a channel surrounded by two channels which have a different pressure, the size and shape of the surrounded channel will be affected by the pressure in each surrounding channel and the volume of aqueous liquid will be modified.

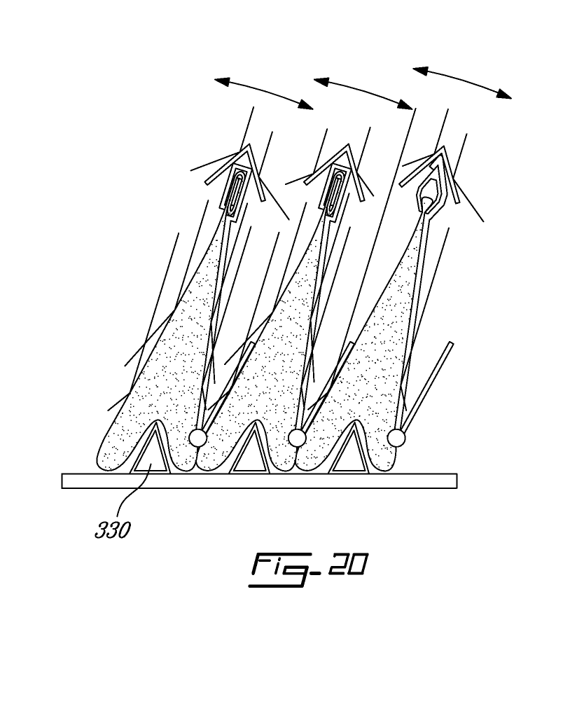

As shown in FIG. 22, a mirror 350 can be affixed to the top of the attachment structure 352 to redirect light towards shaded portions of the V-shaped space between the rows of tubing. It facilitates reflection within the V-shaped channels.

FIG. 23 shows a displacement system 360 for the elongated tube embodiment of FIG. 4. Pivot 306 is fixed and a rod 362 is attached to the top end of the structure 364 at attachment 304. The handle 366 is displaced to allow displacement of the elongated tubes.

As will be readily understood, the elongated tubing can be provided in a compressed S-configuration on the ground such as shown in FIG. 4C. The ground can be wet to improve thermal conduction. The elongated tubing can also be disposed in basins with a low depth which can contain liquid, such as water, for example to increase the thermal conduction.

The embodiments described above are intended to be exemplary only. The scope of the invention is therefore intended to be limited solely by the appended claims.

I/We claim:

1. A sun-tracking light distributor system for use in one of an open-ended system and a closed photo-bioreactor for a photosynthetic culture having an aqueous liquid, comprising:
two light distribution walls, provided at an angle and oriented to converge at a bottom end, the light distribution walls being made of a transparent material allowing sunlight to pass therethrough, the two converging light distribution walls creating an elongated V-shaped channel with an interior space adapted to receive the sunlight and an exterior surface adapted to be partly immersed in said aqueous liquid in use;
a displacement system operatively connected to at least one of the two light distribution walls, the displacement system being adapted to change an orientation of said at least one of the two light distribution walls to track a solar position with respect to at least one axis.

2. The sun-tracking light distributor system of claim 1, wherein a longitudinal direction of the elongated V-shaped channel is provided in an East-West orientation.

3. The sun-tracking light distributor system of claim 1, wherein the photosynthetic culture is in a closed photo-bioreactor, the light distribution walls are facing sidewalls of adjacent elongated tubes, the elongated tubes being adapted to contain the aqueous liquid, the interior space of the V-shaped channel being provided by a spacing between the adjacent elongated tubes.

4. The sun-tracking light distributor system of claim 3, wherein said adjacent elongated tubes are one of adjacent sections of a single elongated tube and adjacent independent elongated tubes.

5. The sun-tracking light distributor system of claim 3, wherein the displacement system includes attachment means connected to said elongated tubes to give an inclination to at least one of said sidewalls.

6. The sun-tracking light distributor system of claim 1, wherein the photosynthetic culture is an open-ended system, the two light distribution walls are sidewalls of a V-shaped transparent structure, said V-shaped transparent structure being adapted to be partly immersed in said aqueous liquid in use.

7. The sun-tracking light distributor system of claim 6, wherein the V-shaped transparent structure has a transparent top surface which closes the V-shaped structure into an inverted-triangle structure.

8. The sun-tracking light distributor system of claim 7, wherein the inverted-triangle structure is at least partly hollow.

9. The sun-tracking light distributor system of claim 7, wherein the inverted-triangle structure has at least one channel formed therein to circulate at least one of air, water, gas, nutrients.

10. The sun-tracking light distributor system of claim 6, wherein the V-shaped structure has a perforated channel for transport and distribution of CO₂.

11. The sun-tracking light distributor system of claim 1, wherein the light distributor walls are curved.

12. The sun-tracking light distributor system of claim 1, wherein the displacement system is adapted to change an inclination of both of the two light distribution walls with a single displacement operation.

13. The sun-tracking light distributor system of claim 7, wherein said transparent top surface is a light concentrating element.

14. A method for distributing light in one of an open-ended photo-bioreactor and a closed photo-bioreactor for a photosynthetic culture, comprising:
providing a sun-tracking light distributor system as claimed in claim 1 in said one of an open-ended photo-bioreactor and a closed photo-bioreactor; and
changing an orientation of at least one of said light distribution walls using said displacement system to allow tracking of the sun.
